SEQUENCE LISTING (7 sequences)

SEQ ID NO: 1 - protein, 1368 aa, artificial sequence [sequence omitted]

SEQ ID NO: 2 - DNA, 4104 bp, artificial sequence
  sequence encoding Cas9
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 3 - protein, 1300 aa, artificial sequence
  FnCpfI
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - DNA, 3900 bp, artificial sequence
  sequence encoding FnCpfI
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 5 - protein, 1082 aa, Geobacillus thermodenitrificans T12
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 6 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1274 aa, Artificial Sequence
  MAD7-NLS
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

(19) United States
(12) Patent Application Publication
(10) Pub. No.: US 2024/0002904 A1
(43) Pub. Date: Jan. 4, 2024

(54) Targeted enrichment using nanopore selective sequencing

(71) Applicant: KEYGENE N.V., AE Wageningen (NL)

(72) Inventors: Theodorus Frank Maria ROELOFS, AE Wageningen (NL); René Cornelis Josephus HOGERS, AE Wageningen (NL)

(73) Assignee: KEYGENE N.V., AE Wageningen (NL)

(21) Appl. No.: 18/038,381

(22) PCT Filed: Nov. 24, 2021
(86) PCT No.: PCT/EP2021/082801

§ 371 Date: May 23, 2023

(30) Foreign Application Priority Data
Nov. 24, 2020 (EP) 20209632.7

(51) Int. Cl. C12Q 1/6806

(52) U.S. Cl. CPC C12Q 1/6806

(57) ABSTRACT

The current invention pertains to a method for sequencing of a target nucleic acid fragment from a nucleic acid sample, comprising the steps of cleaving the nucleic acid sample with a first and a second RNA guided or DNA guided endonuclease complex, preferably a first and a second gRNA-CAS complex, thereby generating the target nucleic acid fragment and at least one non-target nucleic acid fragment. The generated fragments are subsequently contacted with an exonuclease, wherein the exonuclease digests only the non-target nucleic acid fragments. Subsequently said target nucleic acid fragment is sequenced using nanopore selective sequencing. The invention further pertains to the use of the enriched target nucleic acid fragments for nanopore selective sequencing the target nucleic acid fragment.

### FIELD OF THE INVENTION

The present invention is in the field of genetic research, more particular in the field of targeted nucleic acid isolation, e.g. for library preparation for further analysis or processing in genetic research. Disclosed are new methods and compositions for complexity reduction of nucleic acid samples or enrichment of target nucleic acids within nucleic acid samples.

### BACKGROUND OF THE INVENTION

A significant component of genetic research is sequence analysis of defined DNA loci. This can be to genotype known variants, or identify sequence changes or variants. Such analysis often needs to be done in a multiplex fashion, e.g., a specific set of loci needs to be analyzed in a large number of samples. The ideal assay to do this is flexible with regards to the number of samples and loci that need to be screened, is highly accurate, and is amenable to different sequencing platforms. Attempts have been made to provide for assays that comprise an enrichment step but are ideally amplification free. For instance, US2014/0134610 describes a complexity reduction method using type II restriction enzymes to fragment nucleic acids in a sample, followed by ligation of protective adapters and subsequently degrading all non-captured nucleic acid using exonucleases. In WO2016/028887, this method is amended by using a programmable endonuclease, i.e. a CRISPR-endonuclease for fragmenting the nucleic acid in the sample.

CRISPRs (Clustered Regularly Interspaced Short Palindromic Repeats) are loci containing multiple short direct repeats and are found in 40% of the sequenced bacteria and 90% of sequenced archaea. The CRISPR repeats form a system of acquired bacterial immunity against genetic pathogens such as bacteriophages and plasmids. When a bacterium is challenged with a pathogen, a small piece of the pathogen's genome is processed by CRISPR associated proteins (CAS) and incorporated into the bacterial genome between CRISPR repeats. The CRISPR loci are then transcribed and processed to form so called crRNAs which include approximately 30 bps of sequence identical to the pathogen's genome. These RNA molecules form the basis for the recognition of the pathogen upon a subsequent infection and lead to silencing of the pathogen genetic elements through direct digestion of the pathogen's genome. The CAS protein Cas9 is an essential component of the type-II CRISPR-CAS system from S. pyogenes and forms an endonuclease, when combined with the crRNA and a second RNA termed the trans-activating crRNA (tracrRNA), which targets the invading pathogenic DNA for degradation by the introduction of DNA double strand breaks (DSBs) at the position in the genome defined by the crRNA. This type-II CRISPR-Cas9 system has been proven to be a convenient and effective tool in biochemistry that, via the targeted introduction of double-strand breaks and the subsequent activation of endogenous repair mechanisms, is capable of introducing modification in eukaryotic genomes at sites of interest. Jinek et al. (2012, Science 337: 816-820) demonstrated that a single chain chimeric RNA (single guide RNA, sRNA, sgRNA), produced by combining the essential sequences of the crRNA and tracrRNA into a single RNA molecule, was able to form a functional endonuclease in combination with Cas9. Many different CRISPR-CAS systems have been identified from different bacterial species (Zetsche et al. 2015 Cell 163, 759-771; Kim et al. 2017, Nat. Commun. 8, 1-7 Ran et al. 2015. Nature 520, 186-191). Besides CRISPR-CAS systems, in which RNA guides are used to direct an endonuclease to a specific position in a nucleic acid molecule, other endonucleases are known in the art which use DNA or RNA guides (Doxzen et al. 2017, PLOS ONE 12(5): e0177097; Kaya et al. 2016, PNAS vol. 113 no. 15, 4057-4062).

There is still a strong need in the art for a flexible and accurate method for nucleic acid complexity reduction. There is in particular a need in the art for a versatile method to enrich a sample for one or more target nucleic acid fragments, e.g. for subsequent analysis or processing in genetic research.

The present invention, described in detail below, allows for a highly simplified method of library preparation for downstream processing and/or analysis.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

As used herein, the term “adapter” is a single-stranded, double-stranded, partly double-stranded, Y-shaped or hairpin nucleic acid molecule that can be attached, preferably ligated, to the end of other nucleic acids, e.g., to one or both strands of a double-stranded DNA molecule, and preferably has a limited length, e.g., about 10 to about 200, or about 10 to about 100 bases, or about 10 to about 80, or about 10 to about or about 10 to about 30 base pairs in length, and is preferably chemically synthesized. The double-stranded structure of the adapter may be formed by two distinct oligonucleotide molecules that are base paired with one another, or by a hairpin structure of a single oligonucleotide strand. As would be apparent, the attachable end of an adapter may be designed to be compatible with, and optionally ligatable to, overhangs made by cleavage by a restriction enzyme and/or programmable nuclease, may be designed to be compatible with an overhang created after addition of a non-template elongation reaction (e.g., 3′-A addition), or may have blunt ends.

“And/or”: the term “and/or” refers to a situation wherein one or more of the stated cases may occur, alone or in combination with at least one of the stated cases, up to with all of the stated cases.

“Amplification” used in reference to a nucleic acid or nucleic acid reactions, refers to in vitro methods of making copies of a particular nucleic acid, such as a target nucleic acid, or a tagged nucleic acid. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that is hybridizable, but not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Comprising”: this term is construed as being inclusive and open ended, and not exclusive. Specifically, the term and variations thereof mean the specified features, steps or components are included. These terms are not to be interpreted to exclude the presence of other features, steps or components.

“Construct” or “nucleic acid construct” or “vector”: this refers to a man-made nucleic acid molecule resulting from the use of recombinant DNA technology and which can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g., a coding sequence) is integrated downstream of the transcription regulatory sequence. Vectors may comprise further genetic elements to facilitate their use in molecular cloning, such as e.g., selectable markers, multiple cloning sites and the like.

The terms “double-stranded” and “duplex” as used herein, describes two complementary polynucleotides that are base-paired, i.e., hybridized together. Complementary nucleotide strands are also known in the art as reverse-complement.

The term “effective amount,” as used herein, refers to an amount of a biologically active agent that is sufficient to elicit a desired biological effect. For example, in some embodiments, an effective amount of an exonuclease may refer to the amount of the exonuclease that is sufficient to induce cleavage of an unprotected nucleic acid. As will be appreciated by the skilled artisan, the effective amount of an agent may vary depending on various factors such as the agent being used, the conditions wherein the agent is used, and the desired biological effect, e.g. degree of nuclease cleavage to be detected.

“Exemplary”: this terms means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn can be translated into a protein or peptide.

A “guide sequence” is to be understood herein as a sequence that directs an RNA or DNA guided endonuclease to a specific site in an RNA or DNA molecule. In the context of a gRNA-CAS complex, “guide sequence” is further to be understood herein as the section of the sgRNA or crRNA, which is required for targeting a gRNA-CAS complex to a specific site in a duplex DNA.

A gRNA-CAS complex is to be understood herein a CAS protein, also named a CRISPR-endonuclease or CRISPR-nuclease, which is complexed or hybridized to a guide RNA, wherein the guide RNA may be a crRNA and/or a tracrRNA, or a sgRNA.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “nucleotide” includes, but is not limited to, naturally-occurring nucleotides, including guanine, cytosine, adenine and thymine (G, C, A and T, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial, cell free DNA (cfDNA), DNA from a library and/or RNA from a library.

The term “nucleic acid sample” or “sample comprising a nucleic acid” as used herein denotes any sample containing a nucleic acid, wherein a sample relates to a material or mixture of materials, typically, although not necessarily, in liquid form, containing one or more target nucleotide sequences of interest. The nucleic acid sample used as starting material in the method of the invention can be from any source, e.g., a whole genome, a collection of chromosomes, a single chromosome, one or more regions from one or more chromosomes or transcribed genes, and may be purified directly from the biological source or from a laboratory source, e.g., a nucleic acid library. Hence, it follows that the nucleic acid sample used as starting material in the method of the invention, comprising a nucleic acid molecule with the sequence of interest, may further comprise one or more nucleic acid molecules not comprising the sequence of interest. In case of multiple sequences of interest, said multiple sequences may be located on a single or on multiple nucleic acid molecules within the sample. The nucleic acid samples can be obtained from the same individual, which can be a human or other species (e.g., plant, bacteria, fungi, algae, archaea, etc.), or from different individuals of the same species, or different individuals of different species. For example, the nucleic acid samples may be from a cell, tissue, biopsy, bodily fluid, genome DNA library, cDNA library and/or a RNA library.

The term “sequence of interest”, “target nucleotide sequence of interest” and “target sequence” are used interchangeably herein and includes, but is not limited to, any genetic sequence preferably present within a cell, such as, for example a gene, part of a gene, or a non-coding sequence within or adjacent to a gene. The target sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said nucleic acid sequence of interest may be present in a double or a single strand nucleic acid.

The sequence of interest can be, but is not limited to, a sequence having or suspected of having, a polymorphism, e.g. a SNP.

The term “oligonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, 20 to 30, 30 to 40, 40 to 50, 50 to 60, 60 to 70, 70 to 80, 80 to 100, 100 to 150, 150 to 200, or about 200 to 250 nucleotides in length, for example.

“Plant”: this includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grains and the like. Non-limiting examples of plants include crop plants and cultivated plants, such as barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, and zucchini.

The “protospacer sequence” is the sequence that is recognized or hybridizable to a guide sequence within a guide RNA, more specifically the crRNA or, in case of a sgRNA, the crRNA part of the guide RNA, and is located in, at or near the target sequence.

An “endonuclease” is an enzyme that hydrolyses at least one strand of a duplex DNA or a strand of an RNA molecule, upon binding to its target or recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

An “exonuclease” is defined herein as any enzyme that cleaves one or more nucleotides from the end (exo) of a polynucleotide.

“Reducing complexity” or “complexity reduction” is to be understood herein as the reduction of a complex nucleic acid sample, such as samples derived from genomic DNA, cfDNA derived from liquid biopsies, isolated RNA samples and the like. Reduction of complexity results in the enrichment of one or more specific target sequences or target nucleic acid fragments (also denominated herein as target fragments) comprised within the complex starting material and/or the generation of a subset of the sample, wherein the subset comprises or consists of one or more specific target sequences or fragments comprised within the complex starting material, while non-target sequences or fragments are reduced in amount by at least 20%, 30%, 40%, 50%, 60%, 70%, 80%, 90%, 91%, 92%, 93%, 94%, 95%, 96%, 97%, 98% or 99% as compared to the amount of non-target sequences or fragments in the starting material, i.e. before complexity reduction. Reduction of complexity is in general performed prior to further analysis or method steps, such as amplification, barcoding, sequencing, determining epigenetic variation etc. Preferably complexity reduction is reproducible complexity reduction, which means that when the same sample is reduced in complexity using the same method, the same, or at least comparable, subset is obtained, as opposed to random complexity reduction. Examples of complexity reduction methods include for example AFLP® (Keygene N. V., the Netherlands; see e.g., EP 0 534 858), Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al., 1999, The Plant Journal, vol. 20 (6): 719-726) and modifications of SAGE (see e.g. Powell, 1998, Nucleic Acids Research, vol. 26 (14): 3445-3446; and Kenzelmann and Mühlemann, 1999, Nucleic Acids Research, vol. 27 (3): 917-918), MicroSAGE (see e.g. Datson et al., 1999, Nucleic Acids Research, vol. 27 (5): 1300-1307), Massively Parallel Signature Sequencing (MPSS; see e.g. Brenner et al., 2000, Nature Biotechnology, vol. 18:630-634 and Brenner et al., 2000, PNAS, vol. 97 (4):1665-1670), self-subtracted cDNA libraries (Laveder et al., 2002, Nucleic Acids Research, vol. 30(9):e38), Real-Time Multiplex Ligation-dependent Probe Amplification (RT-MLPA; see e.g. Eldering et al., 2003, vol. 31 (23): e153), High Coverage Expression Profiling (HiCEP; see e.g. Fukumura et al., 2003, Nucleic Acids Research, vol. 31(16):e94), a universal micro-array system as disclosed in Roth et al. (Roth et al., 2004, Nature Biotechnology, vol. 22 (4) : 418-426), a transcriptome subtraction method (see e.g. Li et al., Nucleic Acids Research, vol. 33 (16): e136), and fragment display (see e.g. Metsis et al., 2004, Nucleic Acids Research, vol. 32 (16): e127).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained. The term “next-generation sequencing” refers to the so-called parallelized sequencing-by-synthesis or sequencing-by-ligation platforms, e.g., such as currently employed by Illumina, Life Technologies, PacBio and Roche etc. Next-generation sequencing methods may also include nanopore sequencing methods, such as those commercialized by Oxford Nanopore Technologies, or electronic-detection based methods such as Ion Torrent technology commercialized by Life Technologies. Preferably, the next-generation sequencing method is a nanopore sequencing method, preferably a nanopore selective sequencing method.

“Target nucleic acid fragment” or “Target fragment” may be a small or longer stretch, or selected portion of a nucleic acid, single or double stranded, comprising or consisting of a sequence of interest, that is preferably the object of a further analysis or action, such as, but not limited to copying, amplification, sequencing and/or other procedure for nucleic acid interrogation. Prior to the complexity reduction, the target nucleic acid fragment is preferably comprised within a larger nucleic acid molecule, e.g. within a larger nucleic acid molecule present in a sample to be analyzed.

The sequence of interest may be any sequence within a sample nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. In some aspects, a set of target nucleic acid fragments comprising or consisting of one or more sequences of interest are selected to be enriched. Optionally, such set consists of structurally or functionally related target nucleic acid fragments. A target fragment, or target fragments, can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotide such as methylated DNA, and mimetics and combinations thereof. Preferably, the sequence of interest is a small or longer contiguous stretch of nucleotides (i.e. a polynucleotide) of a single-strand DNA strand of duplex DNA, wherein said duplex DNA further comprises a sequence complementary to the target sequence in the complementary strand of said duplex DNA. Duplex DNA consisting of the sequence of interest and its complementary strand is also denominated herein as a target nucleic acid fragment duplex DNA. Preferably, said duplex DNA is genomic DNA (gDNA) and/or cell free DNA (cfDNA).

“Nanopore selective sequencing” is to be understood herein as selectively sequencing of single molecules in real time using nanopore sequencing technology such as from Oxford Nanopore or Ontera, and mapping streaming nanopore current signals or base calls to a reference sequence in order to reject non-target sequences. In response to the data being generated, the sequencer is steered to either pursue sequencing of a nucleic acid, or decides to quit and remove the nucleic acid from the sequencing pore by reversing the polarity of the voltage across the specific pore for a certain short period of time sufficient to eject the non-target molecule and making the nanopore available for a new sequencing read. Examples of Nanopore selective sequencing methods are described in Payne et al., 2020 (Nanopore adaptive sequencing for mixed samples, whole exome capture and targeted panels, Feb. 3, 2020; DOI: 10.1101/2020.02.03.926956) and Kovaka et al. 2020 (Targeted nanopore sequencing by real-time mapping of raw electrical signal with UNCALLED, Feb. 3, 2020; doi: 10.1101/2020.02.03.931923), which are incorporated herein by reference.

### DETAILED DESCRIPTION OF THE INVENTION

The inventors discovered a highly simplified and effective method for high multiplex target enrichments of a nucleic acid sample, by targeting selective fragments with a gRNA-CAS complex followed by removal of non-targeted fragments by exonuclease cleavage as described in WO2020/109412, which is incorporated herein by reference, in combination with nanopore selective sequencing. Surprisingly, using this method, the inventors were capable of drastically enriching the target nucleic acid. At least one benefit of this unexpected enrichment is the efficiency of sequencing, allowing for a substantial increase in number of samples to be sequenced in a particular time span.

In a first aspect, provided is a method for enrichment of at least one target nucleic acid fragment from a sample comprising a nucleic acid molecule. Preferably, the target nucleic acid fragment comprises a sequence of interest. Preferably, said nucleic acid fragment is comprised within the nucleic acid molecule present in the sample prior to the enrichment steps as detailed herein below. Hence preferably, the target nucleic acid fragment is a fragment of the nucleic acid molecule in the sample.

Preferably, the invention pertains to a method for sequencing a target nucleic acid fragment from a sample comprising a nucleic acid molecule, wherein the target nucleic acid fragment comprises a sequence of interest, wherein the method comprises the steps of:
a) providing a sample comprising the nucleic acid molecule, wherein the nucleic acid molecule comprises the sequence of interest
b) cleaving the nucleic acid molecule with at least a first and a second RNA or DNA guided endonuclease complex, thereby generating the target nucleic acid fragment comprising the sequence of interest and at least one non-target nucleic acid fragment;
c) contacting the cleaved nucleic acid molecules obtained in step b) with an exonuclease and allowing the exonuclease to digest the at least one non-target nucleic acid fragment;
d) optionally purifying the (non-digested) target nucleic acid fragment comprising the sequence of interest from the digest obtained in step c); and
e) sequencing the non-digested and optionally purified target nucleic acid fragment by nanopore selective sequencing.

Preferably, the RNA or DNA guided endonuclease complex in step b) is at least one of a gRNA-CAS complex, a gRNA-argonaute complex and a gDNA-argonaute complex. Preferably, the RNA or DNA guided endonuclease complex in step b) is a gRNA-CAS complex.

Preferably, in step c) the at least first and second gRNA-CAS complex are bound to the target nucleic acid fragment.

Preferably, in step c) the at least first and second gRNA-CAS complex remain bound to the target nucleic acid fragment during, or during at least part of, step c).

Preferably, in step c) the target nucleic acid fragment is not digested by the exonuclease, i.e. in step c) the target nucleic acid fragment is protected against exonuclease digestion.

Preferably, in step c) only the one or more non-target nucleic acid fragments are digested by the exonuclease.

In step b) the nucleic acid molecule is cleaved with at least a first and a second gRNA-CAS complex. Optionally, step b) can be further specified in a step of contacting the nucleic acid molecule with the first and second gRNA-CAS complex and a step of allowing the complexes to cleave the nucleic acid molecule. Hence in an embodiment, step b) can be further specified as follows:
b1) contacting the nucleic acid molecule with at least a first and a second gRNA-CAS complex, wherein the gRNA of the first complex guides said first complex to a sequence that is upstream of the sequence of interest, and wherein the gRNA of the second complex guides said second complex to a sequence that is downstream of the sequence of interest; and
b2) allowing the first and second gRNA-CAS complexes to cleave the nucleic acid molecule, wherein at least one cleaved nucleic acid molecule is the target nucleic acid fragment and at least one, preferably two, cleaved nucleic acid molecule(s) is (are) a non-target nucleic acid fragment(s).

Adding exonuclease to the digest of step b), without taking further measures to protect the target nucleic acid fragment, results in enrichment of the said fragment of interest. In other words, no further protection by for instance ligation of inert adapters, is needed to protect the target nucleic acid fragment(s) from exonuclease degradation. Therefore, the method of the invention preferably does not comprise a further step of protecting the target nucleic acid fragment, or the ends of the target nucleic acid fragment, prior to the step of exonuclease treatment. In a preferred embodiment, the method as defined herein is free of adding protective adapters prior the exonuclease treatment. In this context, a protective adapter is to be understood herein as an adapter that is specifically designed to protect the target nucleic acid fragment captured by the adapter for exonuclease digestion. Such adapter preferably protects against exonuclease degradation either by the inclusion of chemical moieties or blocking groups (e.g. phosphorothioate) or by a lack of terminal nucleotides (hairpin or stem-loop adapters, or circularizable adapters).

The method of the invention is e.g. for obtaining sequence information of a particular subset of nucleic acids (fragments) of a nucleic acid sample, preferably in order to analyze one or more target nucleic acids (fragments) within said sample. The method of the invention results in reduction of complexity of the nucleic acid sample used as starting material in step a) of the method of the invention and/or the generation of a subset of one or more target nucleic acid fragments of the nucleic acid sample used as starting material in step a) of the method of the invention.

Therefore, the first aspect of the invention also provides for at least:
i) a method for complexity reduction of a nucleic acid sample comprising a sequence of interest, comprising steps a)-c) and e), and optionally step d), as defined above;
ii) a method for providing a subset of a nucleic acid sample, comprising steps a)-c) and e) and optionally step d) as defined above, wherein said subset comprises one or more target nucleic acid fragments; and
iii) a method for isolating or obtaining a fragment, i.e. a target nucleic acid fragment, comprising a sequence of interest from a nucleic acid molecule comprising said sequence of interest, comprising steps a)-c) and e), and optionally step d), as defined above.

Reducing complexity of a nucleic acid sample finds particular utility in nucleic acid sequencing applications, especially in samples wherein the target nucleic acid fragment is a minor species within a complex sample such as, but not limited to, a genome. Enrichment or complexity reduction substantially decreases the cost of sequencing data generated as the majority of the complex sample is removed prior to sequencing, while the target nucleic acid fragment is selectively retained, therefore a higher percentage of the sequence reads are generated from the sequence of interest.

In preferred embodiments, the enriched target nucleic acid fragments produced by steps a)-d) of the method herein are used in nanopore selective sequencing, wherein during real time sequencing the generated data (either direct current signals or base calls translated from these current signals) is compared to one or more reference sequence(s). In case a set number of nucleotides or amount of signals of the target sequence align with the reference sequence, sequencing will proceed, if not, current is reversed thereby removing the nucleic acid from the pore and making the pore available for sequencing of a new nucleic acid. The set number of nucleotides may be at least the first 50, 60, 70, 80, 90, 100, 150, 200, 250, 300, 350, 400, 450, or 500 nucleotides of the nucleic acid read. The one or more reference sequences may be a multitude of different sequences. Preferably, the reference sequences are at least 50, 60, 70, 80, 90, 92, 93, 94, 95, 96, 97 98, 99 or 100% identical to the sequence of a target nucleic acid fragment obtained in steps a)-c), and optionally step d), of the method of the invention. In an embodiment, the reference sequences are at least 50, 60, 70, 80, 90, 92, 93, 94, 95, 96, 97 98, 99 or 100% identical to a particular subset of the one or more sequences of target nucleic acid fragments obtained in steps a)-c), and optionally step d) of the method of the invention. One of the benefits of the present invention is that in different sequencing runs, different subsets may be sequenced using the same library prepared using steps a)-c), and optionally step d) of the method of the present invention. Preferably, after sequencing the reads obtained in step e) of the method of the invention and comprising the sequences of interest are enriched least 5-, 10-, 15-, 20-, 25-, 30, 35-, 40-, 45-, 50-, 55-, 60-fold over the reads of non-target nucleic acid fragments.

The at least first and second gRNA-CAS complexes are to be understood herein as a CRISPR associated (CAS) proteins, or CRISPR-nucleases, each complexed with a guide RNA. A CRISPR-nuclease comprises a nuclease domain and at least one domain that interacts with a guide RNA. When complexed with a guide RNA, the CRISPR-nuclease is directed to a specific nucleic acid sequence by a guide RNA. The guide RNA interacts with the CRISPR-nuclease as well as with the specific target nucleic acid sequence, such that, once directed to the site comprising the specific nucleic acid sequence via the guide sequence, the CRISPR-nuclease is able to introduce a break at the target site. Preferably, the CRISPR-nuclease is able to introduce a single or double strand break at the target site, in case one or both domains of the nuclease are catalytically active, respectively. The skilled person is well aware of how to design a guide RNA in a manner that it, when combined with a CRISPR-nuclease, effects the introduction of a single- or double-stranded break at a predefined site in the nucleic acid molecule.

CRISPR-nucleases can generally be categorized into six major types (Type I-VI), which are further subdivided into subtypes, based on core element content and sequences (Makarova et al, 2011, Nat Rev Microbiol 9:467-77 and Wright et al, 2016, Cell 164(1-2):29-44). In general, the two key elements of a CRISPR-CAS system complex is a CRISPR-nuclease and a crRNA. CrRNA consists of short repeat sequences interspersed with spacer sequences derived from invader DNA. CAS proteins have various activities, e.g., nuclease activity. Thus, gRNA-CAS complexes provide mechanisms for targeting a specific sequence as well as certain enzyme activities upon the sequence.

Type I CRISPR-CAS systems typically comprise a Cas 3 protein having separate helicase and DNase activities. For example, in the Type 1-E system, crRNAs are incorporated into a multi-subunit effector complex called Cascade (CRISPR-associated complex for antiviral defense) (Brouns et al, 2008, Science 321: 960-4), which specifically binds to duplex DNA and triggers degradation by the Cas3 protein (Sinkunas et al., 2011, EMSO J 30: 1335-1342; Beloglazova et al., 2011, EMBO J 30:616-627).

Type II CRISPR-CAS systems include a signature Cas9 protein, a single protein (about 160 KDa), capable of generating crRNA and specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CAS protein of the type II CRISPR/-CAS system and forms an endonuclease, when combined with the crRNA and a second RNA termed the trans-activating crRNA (tracrRNA), which targets the invading pathogen DNA for degradation by the introduction of DNA double strand breaks (DSBs) at the position in the pathogen genome defined by the crRNA. Jinek et al. (2012, Science 337: 816-820) demonstrated that a single chain chimeric guide RNA (herein “sgRNA) produced by fusing an essential portion of the crRNA and tracrRNA was able to form a functional endonuclease in combination with the Cas9 protein.

Type III CRISPR-CAS systems contain polymerase and RAMP modules. Type III systems can be further divided into sub-types III-A and III-B. Type III-A CRISPR-CAS systems have been shown to target plasmids, and the polymerase-like proteins of Type III-A systems are involved in the specific cleavage of DNA (Marraffini and Sontheimer, 2008, Science 322: 1843-1845). Type III-B CRISPR-CAS systems have also been shown to target RNA (Hale et al, 2009, Cell 139:945-956).

Type IV CRISPR-CAS systems include Csf1, an uncharacterized protein proposed to form part of a Cascade-like complex, though these systems are often found as isolated cas genes without an associated CRISPR array.

A Type V CRISPR-CAS system has recently been described, the Clustered Regularly Interspaced Short Palindromic Repeats from Prevotella and Francisella 1 or CRISPR/Cpf1. Cpf1 genes are associated with the CRISPR locus and coding for an endonuclease that use a crRNA to target DNA. Cpf1 is a smaller and simpler endonuclease than Cas9, which may overcome some of the CRISPR-Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3): 759-771). The type V CRISPR-CAS system preferably includes at least one of Cpf1, C2c1 and C2c3.

A Type VI CRISPR-CAS system may comprise a Cas13a protein, which comprises RNaseA activity. In case the target nucleic acid fragment is RNA, the at least first and second gRNA-CAS complex of the method of the invention may comprise Cas13a, such as, but not limited to Cas13 a from Leptotreichia wadee (LwCas13a) or from Leptotrichia shahii (LshCas13a) such as described in Gootenberg et al., Science. 2017 Apr. 28; 356(6336):438-442.

The first and second gRNA-CAS complexes of the method of the invention may comprise any CRISPR-nuclease as defined herein above. Preferably, at least one of the first and second gRNA-CAS complexes of the method of the invention comprises a Type II CRISPR-nuclease, e.g., Cas9 (e.g., the protein of SEQ ID NO: 1, encoded by SEQ ID NO: 2, or the protein of SEQ ID NO: 5) or a Type V CRISPR-nuclease, e.g. Cpf1 (e.g., the protein of SEQ ID NO: 3, encoded by SEQ ID NO: 4) or Mad7 (e.g. the protein of SEQ ID NO: 6 or 7), or protein derived thereof, having preferably at least about 70%, 80%, 90%, 91%, 92%, 93%, 94%, 95%, 96%, 97%, 98%, or 99% sequence identity to said protein over its whole length.

Preferably, at least one of the first and second gRNA-CAS complexes of the method of the invention comprises a Type II CRISPR-nuclease, preferably a Cas9 nuclease.

The skilled person knows how to prepare the different components of the CRISPR-CAS system, including CRISPR-nuclease. In the prior art, numerous reports are available on its design and use. See for example the recent review by Haeussler et al (J Genet Genomics. (2016)43(5):239-50. doi: 10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

In general, a CRISPR-nuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337: 816-821). A dead CRISPR-nuclease comprises modifications such that none of the nuclease domains shows cleavage activity. The CRISPR-nuclease of at least one of the first and second gRNA-CAS complexes used in the method of the invention may be a variant of a CRISPR-nuclease wherein one of the nuclease domains is mutated such that it is no longer functional (i.e., the nuclease activity is absent), thereby creating a nickase. An example is a SpCas9 variant having either the D10A or H840A mutation. Preferably, the nuclease of the at least one of the first and second gRNA-CAS complexes is not a dead nuclease. Preferably, the CRISPR-nuclease of the first gRNA-CAS complex is either a nickase or (endo)nuclease. Preferably, the CRISPR-nuclease of the second gRNA-CAS complex is either a nickase or (endo)nuclease.

The at least first and second gRNA-CAS complexes of the method of the invention may comprise or consist of a whole Cas9 protein or variant or may comprise a fragment thereof. Preferably such fragment does bind crRNA and tracrRNA or sgRNA, but may lack one or more residues required for nuclease activity.

Preferably, at least one of the first and second gRNA-CAS complex comprises a Cas9 protein. Optionally, both the first and second gRNA-CAS complexes of the method of the invention comprise a Cas9 protein. The Cas9 protein may be derived from the bacteria Streptococcus pyogenes (SpCas9; NCBI Reference Sequence NC_017053.1; UniProtKB—Q99ZW2), Geobacillus thermodenitrificans (UniProtKB—A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase.

According to a preferred embodiment, the programmable nuclease may be derived from Cpf1, e.g., Cpf1 from Acidaminococcus sp; UniProtKB—U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain.

The at least first and second gRNA-CAS complexes further comprise a CRISPR-nuclease associated guide RNA that directs the complex to a defined site in the nucleic acid sample, also named the protospacer sequence. A guide RNA comprises a guide sequence for targeting the gRNA-CAS complex to the protospacer sequence that is preferably near, at or within the sequence of interest in the nucleic acid molecule, and may be a sgRNA or the combination of a crRNA and a tracrRNA (e.g. for Cas9) or a crRNA only (e.g. in case of Cpf1). Optionally, more than one type of guide RNA may be used in the same experiment, for example aimed at two or more different sequences of interest, or even aimed at the same sequence of interest.

It is understood herein that the sequence of interest is present in the nucleic acid sample prior to cleavage with the at least first and second gRNA-CAS complex. Cleavage of the nucleic acid sample results in at least two or more nucleic acid fragments, wherein at least one nucleic acid fragment is a target nucleic acid fragment and at least one nucleic acid fragment is a non-target nucleic acid fragment. Preferably, cleavage of the nucleic acid molecule comprising the sequence of interest with a first and second gRNA-CAS complex results in at least three nucleic acid fragments, wherein at least one nucleic acid fragment is a target nucleic acid fragment and at least two nucleic acid fragments are non-target nucleic acid fragments, i.e. the first and the second gRNA-CAS complex at either side of the sequence of interest generate a double strand break. The target nucleic acid fragment comprises or consists of the sequence of interest. Hence, prior to cleaving the nucleic acid sample, it is clear for the skilled person that the target nucleic acid fragment is encompassed within the nucleic acid sample and the target nucleic acid fragment is released from the nucleic acid sample upon cleavage. The inventors discovered that a nucleic acid fragment cleaved by a gRNA-CAS complex is protected against digestion, preferably exonuclease digestion.

The method of the invention requires that the gRNA of the first gRNA-CAS complex guides said first complex to a sequence in the nucleic acid sample, such that the first gRNA-CAS complex cleaves the nucleic acid molecule within the nucleic acid sample upstream of the sequence of interest, and the gRNA of the second complex guides the second gRNA-CAS complex to a sequence in the nucleic acid sample, such that the second gRNA-CAS complex cleaves the nucleic acid molecule within the nucleic acid sample downstream of the sequence of interest.

Preferably, the gRNA-CAS complex comprises a CRISPR-nuclease that cleaves the nucleic acid within the protospacer sequence. A preferred CRISPR-nuclease is Cas9.

The protospacer sequence bound by the first gRNA-CAS complex can be a sequence in the target nucleic fragment and/or in a non-target nucleic acid fragment. Likewise, the protospacer sequence bound by the second gRNA-CAS complex can be a sequence in the target nucleic fragment and/or in a non-target nucleic acid fragment. Preferably, the protospacer sequence is a sequence that overlaps with the target nucleic fragment and a non-target-nucleic acid fragment, i.e. the cleavage site of the gRNA-CAS complex being within the protospacer sequence.

Preferably, the location of the protospacer sequence is dependent on the CRISPR-nuclease used in the method of the invention. As a non-limiting example, the CRISPR-nuclease SpCAS9 cleaves the nucleic acid within the protospacer sequence. Hence when CAS9 is used in the method of the invention, preferably the protospacer sequence is partly located in the target nucleic acid fragment and partly located in a non-target fragment, i.e. the protospacer sequence is overlapping between the target nucleic acid fragment and a non-target nucleic acid fragment. Preferably, the longest portion of the protospacer sequence remaining after cleavage is part of the target nucleic acid fragment. Hence preferably, the guide sequence of the gRNA of at least one of the first and second gRNA-CAS complex is capable of hybridizing to a protospacer sequence selected from the group consisting of
A) A protospacer sequence comprised in the target nucleic acid fragment;
B) A protospacer sequence comprised in a non-target nucleic acid fragment; and
C) A protospacer sequence overlapping between the target nucleic acid fragment and a non-target nucleic acid fragment.
A) In an embodiment, the guide sequence of the gRNA of at least one of the first gRNA-CAS complex and second gRNA-CAS complex is capable of hybridizing to a sequence that is, or that is part of, the sequence of the target nucleic acid fragment, or a sequence complementary thereof in the opposite strand, e.g. in case the nucleic acid fragment is double stranded. In other words, in this embodiment the protospacer sequence targeted by at least one of the first and second gRNA-CAS complex is, or is located in, a sequence of the target nucleic acid fragment. Preferably, the protospacer sequence targeted by the at least first gRNA-CAS complex is, or is located adjacent to, the 5′-end of the sequence of the target nucleic acid fragment, or a sequence complementary thereof, and preferably the protospacer sequence targeted by the at least second gRNA-CAS complex is, or is located adjacent to, the 3′-end of the sequence of the target nucleic acid fragment, or a sequence complementary thereof. Adjacent, may be directly adjacent, or preferably at a distance of no more than about 1, 2, 3, 4, 5, 6, 7, 8, 9, 10, 20, 30, 40, 50, 60, 70, 80, 90, 100, 500 or 1000 consecutive nucleotides. The number of nucleotides may depend on the CRISPR-nuclease used in the method of the invention.
B) In an embodiment, the guide sequence of the gRNA of at least one of the first gRNA-CAS complex and second gRNA-CAS complex is capable of hybridizing to a sequence that will form, or will form part of, a non-target nucleic acid fragment, or a sequence complementary thereof in the opposite strand, in case the nucleic acid sample is a double stranded nucleic acid. In other words in this embodiment, the protospacer sequence targeted by at least one of the first and second gRNA-CAS complex is located substantially adjacent or directly adjacent to the sequence that will form the target nucleic acid fragment after cleavage. Preferably, the protospacer sequence targeted by the first gRNA-CAS complex substantially flanks, preferably directly flanks, the 5′-end of the target nucleic acid fragment when the fragment is present in the nucleic acid sample, or a sequence complementary thereof. Preferably, the protospacer sequence targeted by the second gRNA-CAS complex flanks, or directly flanks, the 3′-end of the target nucleic acid fragment, when the fragment is present in the nucleic acid sample, or a sequence complementary thereof. Preferably, the distance between the protospacer sequence and respectively the 5′ end or 3′ end of the sequence of the target nucleic acid fragment in the nucleic acid sample, is no more than about 1, 2, 3, 4, 6, 7, 8, 9, 10, 20, 30, 40, 50, 60, 70, 80, 90 or 100 consecutive nucleotides. The number of nucleotides may depend on the CRISPR-nuclease used in the method of the invention.
C) In a preferred embodiment, the guide sequence of at least one of the first gRNA-CAS complex and second gRNA-CAS complex is capable of hybridizing to a sequence that overlaps between the non-target nucleic acid fragment and the target nucleic acid fragment. Preferably, the guide sequence of at least the first or second gRNA-CAS complex is capable of hybridizing to a sequence that overlaps between the 3′ end of a non-target nucleic acid fragment and the 5′ end of the target nucleic acid fragment. Preferably, the guide sequence of at least the first or second gRNA-CAS complex is capable of hybridizing to a sequence that overlaps between the 5′ end of a non-target nucleic acid fragment and the 3′ end of the target nucleic acid fragment. In other words in this embodiment, preferably the protospacer sequence targeted by at least the first or second gRNA-CAS complex overlaps between the 3′-end of a non-target nucleic acid fragment and the 5′-end of the target nucleic acid fragment when said fragments are present in the nucleic acid sample, i.e. prior to cleavage of the nucleic acid sample.

As a non-limiting example, a SpCas9 may cleave within a 20 nt protospacer sequence in between position 3 and 4. As a result the target nucleic acid fragment at its 3′-end may comprise 3 nt of the protospacer sequence and a non-target nucleic acid fragment at its 5′-end may comprise 17 nt of the protospacer sequence. Likewise if the protospacer sequence is on the complementary strand, the target nucleic acid fragment at its 3′-end may comprise 17 nt of the protospacer sequence and a non-target nucleic acid fragment at its 5′-end may comprise 3 nt of the protospacer sequence. Hence in the example wherein the protospacer sequence is 20 consecutive nucleotides, 1, 2, 3, 4, 5, 6, 7, 8, 9, 10, 11, 12, 13, 14, 15, 16, 17, 18 or 19 nucleotides of the protospacer sequence may be present in the 3′-end of a non-target nucleic acid fragment and respectively 19, 18, 17, 16, 15, 14, 13, 12, 11, 10, 9, 8, 7, 6, 5, 4, 3, 2 or 1 nucleotide of the protospacer sequence may be present in the 5′-end of the target sequence, depending on the type of CRISPR-nuclease used in the method of the invention.

Preferably the protospacer sequence targeted by at least the first or second gRNA-CAS complex overlaps between the 5′-end of a non-target nucleic acid fragment and the 3′-end of the target nucleic acid fragment when said fragments are present in the nucleic acid sample, i.e. prior to cleavage of the nucleic acid sample. As a non-limiting example wherein the protospacer sequence is 20 nucleotides, 1, 2, 3, 4, 5, 6, 7, 8, 9, 10, 11, 12, 13, 14, 15, 16, 17, 18 or 19 nucleotides of the protospacer sequence may be present in the 5′-end of the non-target nucleic acid fragment and respectively 19, 18, 17, 16, 15, 14, 13, 12, 11, 10, 9, 8, 7, 6, 5, 4, 3, 2 or 1 nucleotide of the protospacer sequence may be present in the 3-end of the target sequence, depending on the type of CRISPR-nuclease used in the method of the invention.

In a preferred embodiment, at least one of the first and second gRNA-CAS complex binds to a sequence within the target nucleic acid fragment. Preferably, both the first and second gRNA-CAS complex bind to a sequence within the target nucleic acid fragment.

Alternatively or in addition, at least one of the first and second gRNA-CAS complex binds to a sequence within a non-target nucleic acid fragment. Preferably, both the first and second gRNA-CAS complex bind to a sequence within a non-target nucleic acid fragment.

Alternatively or in addition, at least one of the first and second gRNA-CAS complex binds to a sequence that overlaps between the target nucleic acid fragment and a non-target nucleic acid fragment. Preferably, both the first and second gRNA-CAS complex bind to a sequence that overlaps between the target nucleic acid fragment and a non-target nucleic acid fragment.

In a preferred embodiment, at least one of the first and second gRNA-CAS complex remains bound to respectively the 5′-end or 3′-end of the target nucleic acid fragment after cleavage. Preferably, at least one gRNA-CAS complex remains bound to the 5′-end of the target nucleic acid fragment and one gRNA-CAS complex remains bound to the 3′-end of the target nucleic acid fragment after cleavage. Put differently, there is preferably a gRNA-CAS complex flanking both sides of the target nucleic acid fragment.

As the gRNA-CAS complex, apart from a protospacer sequence, requires a protospacer adjacent motif (PAM) sequence for recognition, the gRNA should be designed such that the targeted protospacer sequence is adjacent to such PAM sequence, depending on the gRNA-CAS complex used. The PAM sequence is essential for the CRISPR/Cas endonuclease activity, is relatively short, and is therefore usually present multiple times in any given sequence of some length. For instance the PAM motif of the S. pyogenes Cas9 protein is NGG, which ensures that for any given genomic sequence multiple PAM motifs are present and so many different guide RNAs can be designed. In addition, guide RNAs can also be designed targeting the opposite strands of the same double strand sequence. The sequence immediately adjacent to the PAM is incorporated into the guide RNA. This can differ in length depending upon the CRISPR-CAS complex being used. For instance, the optimal length for the targeting sequence in the Cas9 sgRNA is 20 nt. Depending on the CRISPR/Cas endonuclease being used, the complex then induces nicks in both of the DNA strands at varying distances from the PAM. For instance the S. pyogenes Cas9 protein introduces nicks in the both DNA strands 3 bps upstream from the PAM sequence to create a blunt DNA DSB. Depending on e.g. the gRNA-CAS complex used, the PAM site used to cleave the nucleic acid sample may be present in either the generated target nucleic acid fragment or in a generated non-target nucleic acid fragment.

Preferably, the sequence of interest in the nucleic acid sample is flanked by or comprises, preferably near the ends of the sequence of interest, a PAM sequence known for interacting with the CRISPR-system nuclease of the complex as defined herein (e.g. see Ran et al 2015, Nature 520:186-191). In addition or alternatively, the PAM sequence preferably flanks the protospacer sequence targeted by at least one of the first and second gRNA-CAS complex. In case the CAS protein in the gRNA-CAS complex is S. pyogenes Cas9 (SpCas9) endonuclease, preferably the sequence of interest is flanked by a protospacer sequence at either end of the sequence of interest, which are subsequently flanked by the PAM sequences, i.e. the nucleic acid molecule in the method of the invention preferably comprises the following elements in a sequential order: non-target sequence-PAM-protospacer-sequence of interest-protospacer-PAM-non-target sequence. Preferably, in case the CAS is a SpCas9 endonuclease, the guide RNAs of the respective first and second gRNA-CAS complex are designed such that the PAM sequences end up in the non-target fragments after cleavage by the first and second gRNA-CAS complexes, while the largest portion of the protospacer sequence ends up in the target nucleic acid fragment. In an optional embodiment, in step b1) of the method of the invention, the nucleic acid molecule is contacted with at least two gRNA-CAS complexes, i.e. the first gRNA-CAS complex and a third gRNA-CAS complex, wherein the guides of both the first and third complex guide the complex to a sequence that is upstream of the sequence of interest, and at least two gRNA-CAS complexes, i.e. the second gRNA-CAS complex and a fourth gRNA-CAS complex, wherein the guides of both the second and fourth complex guide the complex to a sequence that is downstream of the sequence of interest. Within this embodiment, the protospacer sequences of the first and third guide RNA are designed to be preferably about 20-80, about 40-60 or about 50 nucleotides apart. Likewise, the protospacer sequences of the second and fourth guide RNA are designed to be preferably about 20-80, about 40-60 or about 50 nucleotides apart. Preferably, at least one of the first and third gRNA-CAS complex and at least one of the second and fourth gRNA-CAS complex effectively bind and preferably subsequently cleave the nucleic acid molecule in the method of the invention in step b2). Within this embodiment, the CAS protein of the first, second, third and fourth gRNA-CAS complex may be a SpCas9 endonuclease. This embodiment is in particular useful in case of sequence variety, for example in case of genomes from different individuals. Designing two gRNA-CAS complexes for cleavage at either end of the fragment increases the chance of successful cleavage. The first and third gRNA-CAS complex may be designed in such a way that they bind to the nucleic acid molecule in the same orientation relative to the sequence of interest. Likewise, the second and fourth gRNA-CAS complex may be designed in such a way that they bind to the nucleic acid molecule in the same orientation relative to the sequence of interest.

For instance, if said CRISPR-nuclease is S. pyogenes Cas9, the PAM sequence may have a sequence of 5′-NGG-3′. For instance, for Geobacillus thermodenitrificans T12 Cas9 (e.g. see WO2016/198361) the PAM sequence may have a sequence of 5′-NNNNCNNA-3′. Further known PAM sequences for Cas9 endonucleases are: Type IIA 5′-NGGNNNN-3′ (Streptococcus pyogenes), 5′-NNGTNNN-3′ (Streptococcus pasteurianus), 5′-NNGGAAN-3′ (Streptococcus thermophilus), 5′-NNGGGNN-3′ (Staphylococcus aureus), and Type IIC 5′-NGGNNNN-3′ (Corynebacterium difteriae), 5′-NNGGGTN-3′ (Campylobacter Ian), 5′-NNNCATN-3′ (Parvobaculum lavamentivorans) and 5′-NNNNGTA-3′ (Neisseria cinerea). The person skilled in the art is therefore able to design gRNAs in order to fragment the target sequence from the nucleic acid of the sample.

Molecules suitable as crRNA and tracrRNA for use as gRNA in a gRNA-CAS complex are well known in the art (see e.g., WO2013142578 and Jinek et al., Science (2012) 337, 816-821).

In an embodiment, at least one of the crRNAs comprises a sequence that can hybridize to or near a sequence of interest, preferably a sequence of interest as defined herein. Therefore preferably, at least one of the crRNAs comprises a nucleotide sequence that is fully complementary to a sequence in the sequence of interest i.e. the sequence of interest comprises a protospacer sequence.

In an embodiment, at least one of the crRNAs comprises a sequence that can hybridize to or near the complement of a sequence of interest, preferably a sequence of interest as defined herein. Therefore preferably, at least one of the crRNAs comprises a nucleotide sequence that has full sequence identity with, or with a part of, the sequence of interest.

Preferably, the crRNA, or crRNAs, is/are also capable of complexing with the tracrRNA. At least one of the crRNAs used in the method of the invention can comprise or consist of non-modified or naturally occurring nucleotides. Alternatively or in addition, the at least one crRNA can comprise or consist of modified or non-naturally occurring nucleotides, preferably such chemically modified nucleotides are for protecting the crRNA against degradation. In an embodiment, at least two or all cRNAs used in the method of the invention can comprise or consist of modified or non-naturally occurring nucleotides.

In an embodiment of the invention, the at least one crRNA comprises ribonucleotides and non-ribonucleotides. The at least one crRNA can comprise one or more ribonucleotides and one or more deoxyribonucleotides.

The at least one crRNA may comprise one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-O-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

The at least one crRNA may be chemically modified by incorporation of 2′-O-methyl (M), 2′-O-methyl 3′phosphorothioate (MS), 2′-O-methyl 3′thioPACE (phosphonoacetate) (MSP), or a combination thereof, at one or more terminal nucleotides. Such chemically modified crRNAs can comprise increased stability and/or increased activity as compared to unmodified crRNAs. (Hendel et al, 2015, Nat Biotechnol. 33(9); 985-989). In certain embodiments, the at least one crRNA comprises ribonucleotides in a region that hybridizes to a protospacer sequence. In an embodiment of the invention, deoxyribonucleotides and/or nucleotide analogues can be incorporated in the engineered crRNA structures, such as, without limitation, in the sequence hybridizing to the protospacer sequence, in the sequence interacting with the tracrRNA or in between these sequences.

Alternatively or in addition, the chemically modified nucleotides can be located 5′ and/or 3′ of the sequence hybridizing to the protospacer sequence. The chemically modified sequences can further be located 5′ and/or 3′ of the sequence interacting with the tracrRNA.

In a preferred embodiment, the length of at least one of the crRNAs can be at least about 15, 20, 25, 32, 34, 36, 38, 40, 42, 44, 46, 48, 50, 52, 54, 56, 58, 60, 65, 70, 75, 80, 85, 90, 95, 100 or more nucleotides in length. In some preferred embodiments, at least one of the crRNAs is less than about 75, 45, 40, 35, 30, 25 or about 20 nucleotides in length. Preferably, the length of the crRNAs used in the method of the invention is about 20-100, 25-80, 30-60 or about 35-50 nucleotides in length.

The part of the crRNA sequence that is complementary to the protospacer sequence is designed to have sufficient complementarity with the protospacer sequence to hybridize with the protospacer sequence and direct sequence-specific binding of a complexed nuclease. The protospacer sequence is preferably adjacent to a protospacer adjacent motif (PAM) sequence, which PAM sequence may interact with the CRISPR nuclease of the RNA-guided CRISPR-system nuclease complex as defined herein. For instance, in case the CRISPR nuclease is S. pyogenes Cas9, the PAM sequence preferably is 5′-NGG-3′, wherein N can be any one of T, G, A or C. The skilled person is capable of engineering the crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least about 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 75, or more nucleotides in length. In some preferred embodiments, a sequence complementary to the DNA target sequence is less than about 75, 50, 45, 40, 35, 30, 25, 20 nucleotides in length. Preferably, the length of the sequence complementary to the DNA sequence is at least 17 nucleotides. Preferably the complementary crRNA sequence is about 10-30 nucleotides in length, about 17-25 nucleotides in length or about 15-21 nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides.

The part of the crRNA that interacts with the tracrRNA is designed to be sufficiently complementary to the tracrRNA to hybridize to the tracrRNA, and direct the complexed nuclease to the protospacer sequence. Preferably, the complementarity between this part of a crRNA sequence and its corresponding part in the tracrRNA, when optimally aligned using a suitable alignment algorithm, is at least about 50%, 60%, 70%, 80%, 85%, 90%, 91%, 92%, 93%, 94%, 95%, 96%, 97%, 98% or 100%. The part of the crRNA that interacts with the tracrRNA is preferably at least about 5, 10, 15, 20, 22, 25, 30, 35, 40, 45 or more nucleotides in length. In some preferred embodiments, the part of the crRNA that interacts with the tracrRNA is less than about 60, 55, 50, 45, 40, 35, 30 or 35 nucleotides in length. Preferably, the part of the crRNA that interacts with the tracrRNA is about 5-40, 10-35, 15-30, 20-28 nucleotides in length. Preferably, the length of the part that interacts with the tracrRNA is 15, 16, 17, 18, 19, 20, 21, 22, 23, 24, 25, 26, 27, 28, 29, 30, 31, 32, 33, 34 or 35 nucleotides.

In an embodiment, the at least first and second gRNA-Cas complex used in the method of the invention comprises respectively a first and a second crRNA. The first and second gRNA-CAS complex however may comprise the same tracrRNA.

Preferably the tracrRNA, comprises one or more structural motifs that can interact with the CRISPR-system nuclease of the complex as defined herein. Preferably, the tracrRNA is also capable of interacting with the crRNA as defined herein. The tracrRNA and the crRNA may hybridize through base-pairing between the crRNA and the tracrRNA. The tracrRNA preferably is capable of forming a complex with the CRISPR-system nuclease and the crRNA. The crRNA is capable of complexing with the tracrRNA and can hybridize with a target sequence, thereby directing the nuclease to the target sequence.

The tracrRNA may comprise one or more stem-loop structures, such as 1, 2, 3 or more stem loop structures.

The tracrRNA can comprise or consist of non-modified or naturally occurring nucleotides. Alternatively or in addition, the tracrRNA can comprise or consist of modified or non-naturally occurring nucleotides, preferably such chemically modified nucleotides are for protecting the tracrRNA against degradation.

In an embodiment of the invention, the tracrRNA comprises ribonucleotides and non-ribonucleotides. The tracrRNA can comprise one or more ribonucleotides and one or more deoxyribonucleotides.

The tracrRNA may comprise one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-O-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

The tracrRNA may be chemically modified by incorporation of 2′-O-methyl (M), 2′-O-methyl 3′phosphorothioate (MS), 2′-O-methyl 3′thioPACE (phosphonoacetate) (MSP), or a combination thereof, at one or more terminal nucleotides. Such chemically modified tracrRNAs can comprise increased stability and/or increased activity as compared to unmodified tracrRNAs. (Hendel et al, 2015, Nat Biotechnol. 33(9); 985-989). In certain embodiments, a tracrRNA comprises ribonucleotides in a region that interacts with the crRNA.

In an embodiment of the invention, deoxyribonucleotides and/or nucleotide analogues can be incorporated in the engineered tracrRNA structures, such as, without limitation, in the sequence that interacts with the crRNA, in the sequence interacting with the CRISPR-system nuclease or in between these sequences.

Alternatively or in addition, the chemically modified nucleotides can be located 5′ and/or 3′ of the sequence interacting with the crRNA. The chemically modified sequences can further be located 5′ and/or 3′ of the sequence interacting with the CRISPR-system nuclease.

In a preferred embodiment, the length of the tracrRNA can be at least about 25, 30, 35, 40, 45, 50, 60, 65, 70, 72, 75, 80, 85, 90, 95, 100, 110, 120, 130, 140, 150 or more nucleotides in length. In some preferred embodiments, the tracrRNA is less than about 200, 180, 160, 140, 120, 100, 95, 90, 85, 80 or 75 nucleotides in length. Preferably, the length of the tracrRNA is bout 30-120, 40-100, 50-90 or about 60-80 nucleotides in length.

The part of the tracrRNA sequence that interacts with the CRISPR-system nuclease is designed to be sufficient to direct the complexed nuclease to the target sequence. The part of the tracrRNA sequence that interacts with the CRISPR-system nuclease may be at least about 20, 25, 30, 35, 40, 45, 50, 55, 60, 70, 72, 75, 80, 85, 90, 95, 100 or more nucleotides in length. In some preferred embodiments, the sequence interacting with the CRISPR-system nuclease is less than about 120, 100, 80, 72, 70, 60, 55, 50, 40, 30 or 20 nucleotides in length. Preferably, the part of the tracrRNA sequence that interacts with the CRISPR-system nuclease is about 20-90, 30-85, 35-80, 40-75 or 50-72 nucleotides in length. Preferably, the part of the tracrRNA that interacts with the CRISPR-system nuclease is about 40, 42, 44, 46, 48, 50, 52, 54, 56, 58, 60, 62, 64, 66, 68, 70, 72, 74 or 76 nucleotides in length.

The part of the tracrRNA that interacts with the crRNA is designed to be sufficiently complementary to the crRNA to hybridize to the crRNA, and direct the complexed nuclease to the target sequence. Preferably, the complementarity between this part of a tracrRNA sequence and its corresponding part in the crRNA, when optimally aligned using a suitable alignment algorithm, is at least about 50%, 60%, 70%, 80%, 85%, 90%, 91%, 92%, 93%, 94%, 95%, 96%, 97%, 98% or 100%. The part of the tracrRNA that interacts with the crRNA is preferably at least about 5, 10, 15, 20, 22, 25, 30, 35, 40, 45 or more nucleotides in length. In some preferred embodiments, the part of the tracrRNA that interacts with the crRNA is less than about 60, 55, 50, 45, 40, 35, 30 or 35 nucleotides in length. In a preferred embodiment, the part of the tracrRNA that interacts with the crRNA is about 5-40, 10-35, 15-30, 20-28 nucleotides in length. Preferably, the length of the part that interacts with the crRNA is 15, 16, 17, 18, 19, 20, 21, 22, 23, 24, 25, 26, 27, 28, 29, 30, 31, 32, 33, 34 or 35 nucleotides.

Preferably, the crRNA and tracrRNA are linked to together to form a sgRNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

Preferably, the gRNA of the at least first and second gRNA-CAS complexes are designed such that upon incubation of the nucleic acid sample with the at least first and second gRNA-CAS complexes, the target nucleic acid fragment comprised within a nucleic acid from the nucleic acid sample is cut out of the said nucleic acid. In addition, preferably the first gRNA is designed such that the first gRNA-CAS complex is bound to the target nucleic acid fragment after cleavage of the nucleic acid sample. In addition preferably the second gRNA is designed such that the second gRNA-CAS complex is bound to the target nucleic acid fragment after cleavage of the nucleic acid sample. Preferably, the target nucleic acid fragment when present in the nucleic acid sample is flanked by at least one non-target nucleic acid fragment. Preferably, the target nucleic acid fragment when present in the nucleic acid sample is flanked on both sides with a non-target nucleic acid fragment, i.e. one non-target nucleic acid fragment is present directly 5′ of the target nucleic acid fragment and one non-target nucleic acid fragment is present directly 3′ of the target nucleic acid fragment.

Preferably, at least one of the first and second gRNA-CAS complexes of the method of the invention comprises a sgRNA for targeting the CRISPR-nuclease, preferably Cas9, to a sequence in the target nucleic acid fragment. Optionally, both the first and second gRNA-CAS complexes of the method of the invention comprise a sgRNA for targeting the respective first or second gRNA-CAS complex to the sequences in the target nucleic acid fragment. Preferably, at least one of the first and second gRNA-CAS complexes of the method of the invention comprises a sgRNA for targeting the CRISPR-nuclease, preferably Cas9, to a sequence adjacent, preferably directly adjacent, to the target nucleic acid fragment, when the fragment is comprised within the nucleic acid sample. Optionally, both the first and second gRNA-CAS complexes of the method of the invention comprise a sgRNA for targeting the respective first or second gRNA-CAS complex to the sequences adjacent, preferably directly adjacent, to the target nucleic acid fragment, wherein the target nucleic acid is comprised in the nucleic acid sample.

Preferably, at least one of the first and second gRNA-CAS complexes of the method of the invention comprises a sgRNA for targeting the CRISPR-nuclease, preferably Cas9, to a sequence overlapping between the target nucleic acid fragment and a non-target nucleic acid fragment, when the fragments are comprised within the nucleic acid sample. Optionally, both the first and second gRNA-CAS complexes of the method of the invention comprise a sgRNA for targeting the respective first or second gRNA-CAS complex to the sequences overlapping between the target nucleic acid fragment and a non-target nucleic acid fragment, wherein the target nucleic acid is comprised in the nucleic acid sample. Optionally, both the first and second gRNA-CAS complexes of the method of the invention comprise a sgRNA for targeting the respective first or second gRNA-CAS complex to respectively a sequence overlapping between the 5′-end of target nucleic acid fragment and the 3′-end of a non-target nucleic acid fragment and to a sequence overlapping between the 3′-end of target nucleic acid fragment and the 5′-end of a non-target nucleic acid fragment, when the target nucleic acid is comprised in the nucleic acid sample.

Alternatively, at least one of the first and second gRNA-CAS complexes of the method of the invention comprise a dual guide RNA for targeting the CRISPR-nuclease, preferably Cas9, to a sequence in the nucleic acid sample, i.e. a protospacer sequence present in the target nucleic acid fragment or present in a non-target nucleic acid fragment. A dual guide RNA (dgRNA) is to be understood herein as comprising or consisting of a crRNA and tracrRNA as separate but preferably hybridized molecules. Optionally, both the first and second gRNA-CAS complexes of the method of the invention comprise a dgRNA for targeting the respective first or second gRNA-CAS complex to the protospacer sequences.

Preferably, the at least one of the first and second gRNA-CAS complexes is capable of inducing a double strand break (DSB). Preferably both the first and second gRNA-CAS complexes is capable of inducing a double strand break (DSB) in the nucleic acid sample.

Alternatively, at least one of the first and second gRNA-CAS complexes is a nickase, indicated herein as a first or second gRNA-CAS-nickase complex, which is capable of nicking only one strand of a duplex DNA. In such embodiment of the invention, in step b) an additional, i.e. third, gRNA-CAS complex is added which is capable of nicking the complementary strand of the duplex DNA at substantially the complementary position nicked by the first or second gRNA-CAS-nickase complex. Nicking the substantially complementary position preferably results in a double stranded, i.e. blunt or staggered, break in the nucleic acid sample.

As a non-limiting example, the protospacer sequence of the e.g. third, gRNA-CAS-nickase is preferably a sequence in the complementary strand that is complementary to the protopospacer sequence targeted by the first gRNA-CAS-nickase complex, or a sequence within shifted about 1, 2, 3, 4, 5, 6, 7, 8, 9, 10, 15, 20, 25, or 30 nucleotides in the upstream or downstream direction of the complementary strand. For instance, in case the first gRNA-CAS complex is a gRNA-CAS-nickase complex, a third gRNA-CAS-nickase complex can be added in step b, resulting in a double strand break induced at one side of the sequence of interest by said first and third gRNA-CAS-nickase complexes, which may be blunt ended, in case the exact opposite positions are nicked by said first and third complexes, or staggered in case the positions nicked by said first and third complexes are not exactly opposite. Likewise, both the use of second and a further, e.g. a fourth, gRNA-CAS-nickase complex in addition to said first and third gRNA-CAS-nickase complexes, may result in two blunt or staggered ends of the target nucleic acid fragment obtained in step b) of the method of the invention. In some instances, it may be desired to create a staggered end at one or both ends of the target nucleic acid fragment produced by step b of the method of the invention, for instance, in case of a subsequent directed adapter ligation.

Step b) of the method of the invention may be performed by incubating the at least first and second gRNA-CAS complex and the nucleic acid sample together at conditions and time suitable for the gRNA-CAS complexes to induce at least a single strand break, optionally a double strand break, such as, but not limited to, the conditions detailed in the Examples provided herein. Optionally, the incubation is performed between about 1 min to about 18 hours, preferably about 60 minutes, at about 10-90° C., preferably about 37° C.

Target nucleic acid fragments cleaved by gRNA-CAS complexes are protected against exonuclease treatment. Therefore, directly after cutting the target nucleic acid fragment from a nucleic acid, exonuclease can be added to digest the non-target nucleic acid or acids. The target nucleic acid fragment is protected from degradation, while the non-protected fragments are degraded, resulting in enrichment or complexity reduction of the target fragment. Therefore, the method of the invention takes the approach of removal of the undesired (non-target) part of the nucleic acid sample instead of removing the portion of interest, thereby circumventing complex affinity selection schemes.

The exonuclease may be exonuclease I, III, V, VII, VIII, or related enzyme, or any combination thereof. Exonuclease III recognizes nicks and extend the nick to a gap until a piece of ssDNA is formed. Exonuclease VII can degrade this ssDNA. Exonuclease I also degrades ssDNA. ExoIII and ExoVII is a preferred combination of exonucleases for use in step c) of the method of the invention.

Exonuclease V is capable of degrading ssDNA and dsDNA in both 3′ to 5′ and in 5′ to 3′ direction. Therefore in a preferred embodiment, the exonuclease in step c) of the method of the invention is an exonuclease that is capable of degrading ssDNA and dsDNA in both 3′ to 5′ and in 5′ to 3′ direction, preferably exonuclease V.

Further information on methods for degrading non-target sequences is provided in U.S. Patent Publication No. 2014/0134610, which is incorporated herein by reference in its entirety for all purposes.

In addition, an endonuclease, i.e. a restriction enzyme, may be used for degradation of the non-protected fragments either together, prior, after, or any combination thereof, the exonuclease digestion of step c) of the method of the invention. It is to be understood herein that restriction enzymes for use in the method of the invention preferably are selected depending on the one or more target sequences of interest enriched using the method of the invention, as preferably the restriction enzyme or enzymes should not have a recognition site that is present within the one or more target sequences of interest, but preferably should have a recognition site that is present at one or more locations in the remainder of the nucleic acid of the sample, i.e. in one or more non-target nucleic acid fragments. The benefit of restriction enzyme digestion prior to the exonuclease treatment of step c) of the method of the invention, or even prior to cleavage reaction of step b), is that such digestion results in fragments that, if not protected by gRNA-CAS complexes, are more easily digested by exonucleases in step c).

Step c), and the optional endonuclease step, is performed at conditions and time sufficient for the exonucleases (and optionally endonucleases) to degrade substantially all non-protected fragments, such as, but not limited to, the conditions detailed in the Examples provided herein. Preferably, step c) is performed at conditions and time sufficient for the exonucleases (and optionally endonucleases) to degrade all non-protected fragments. Step c) is preferably performed for about 1 minute to about 12 hours, preferably 30 min, at about 10-90° C., preferably about 37° C.

After step c), the exonuclease, and optional endonuclease, can be inactivated by, for example, but not limited to, at least one of a Proteinase, e.g. Proteinase K, treatment or heat inactivation. Such techniques are standard in the art and the skilled person straightforwardly understands how to inactivate an exonuclease and optionally an endonuclease. A preferred inactivation step is heating the sample at a temperature of about 50-90° C., preferably about 75° C., for about 1-120 minutes, preferably about 10 minutes. Preferably, the inactivation step is between step c) and d) of the method of the invention.

After step c) of the method of the invention, the sample enriched with one or more target nucleic acid fragments may be subjected to a purification step, e.g., an AMPure bead-based purification process, to remove complexes, enzymes, free nucleotides, possible free adapters, and possible small, non-target, nucleic acid fragments. The target nucleic acid fragments may be recovered after purification and subjected to further processing and/or analysis, such as single-molecule sequencing.

The method of the invention may further comprise a size-selection step. Optionally, the size-selection step is performed prior to step b), between step b) and c), or after step c) of the method of the invention.

The length of the target nucleic acid fragment can vary, but is preferably at least 200, 500, 1000, 3000, 5000, 7000, 10,000, 15,000, or 20,000 (up to at least 100,000) bases in length. The length depends primarily on the intended use, and in some optional embodiments is based upon the average read length of the specific sequencing technique to be used.

It is to be understood herein that an effective amount of components is used in the method of the invention. For instance, the at least first and second gRNA-CAS complex added in step b) is provided in an amount sufficient to induce cleavage of the one or more nucleic acid molecules in a sample. In addition, an exonuclease added in step c) is applied in an amount that is sufficient to degrade at least about 75%, 80%, 85%, 90%, 95%, or 100% of the non-target nucleic acid fragments within the sample or starting material.

The method of the invention may comprise one or more purification steps, preferably after step c) as defined herein. An optional purification step is a proteinase K treatment. Alternatively or in addition, said purification may comprise the following steps:
I. exposing the digested nucleic acid sample obtained after step (c) to one or more solid supports that specifically and effectively bind the one or more target nucleic acid fragments; and optionally,
II. washing the one or more solid supports and eluting the target nucleic acid fragments from the one or more solid supports.
   The one or more solid supports may be, but not limited to, Ampure beads. As after purification, at least one isolated target nucleic acid fragment is obtained, the method as defined herein may also be regarded as a method for isolation of one or more target nucleic acid fragments from a nucleic acid sample.

The method of the invention may be followed by a step of sequencing one or more target nucleic acid fragments. The method as defined herein may therefore also be also regarded as a method for sequencing one or more target nucleic acid fragments from a nucleic acid sample.

Optionally, the method of the invention further comprises an amplification step. Preferably, this amplification is performed after the exonuclease treatment, i.e. after the step c) as defined herein. Amplification can be done by PCR or by any amplification method known in the art.

The method of the invention may also comprise a step of ligating one or more adapters to the target nucleic acid fragment. Preferably, such adapter ligation is performed after step c) as defined herein. These one or more adapters may comprise functional domains, preferably selected from the group consisting of a restriction site domain, a capture domain, a sequencing primer binding site, an amplification primer binding site, a detection domain, a barcode sequence, a transcription promoter domain and a PAM sequence, or any combination thereof. The barcode can be, but is not limited to, a sample barcode, or a unique molecular identifier (UMI).

In particularly preferred embodiments, the one or more adapters are sequencing adapters, e.g. comprise a functional domain that allows for Oxford Nanopore Technologies or Ontera sequencing.

Depending on the adapter design, the adapters may be a, single-stranded, double-stranded, partly double-stranded, Y-shaped, hairpin or circularizable adapters. Optionally, one or more adapters may be used. Optionally, one or more sets of two adapters may be used, wherein a first adapter of a set is aimed to be ligated at the 5′ end side of the target nucleic acid fragment, and the second adapter of set is aimed to be ligated at the 3′ end side of the target nucleic acid fragment. Preferably, the first and second adapter within a set each comprise compatible primer binding sequences, such that adapter ligated fragments are ready to be either amplified using a compatible primer pair or sequenced.

In a preferred embodiment, the method of the invention is free of amplification and/or cloning steps. Reduction of amplification steps is beneficial, as epigenetic information (e.g., 5-mC, 6-mA, etc.) will get lost in amplicons. Further amplification can introduce variations in the amplicons (e.g., via errors during amplification) such that their nucleotide sequence is not reflective of the original sample. Similarly, cloning of a target region into another organism often does not maintain modifications present in the original sample nucleic acid, so in preferred embodiments target sequences to be enriched for further analysis are typically not amplified and/or cloned in the methods herein.

Stem-loop or hairpin adapters are single-stranded, but their termini are complementary such that the adapter folds back on itself to generate a double-stranded portion and a single-stranded loop. A stem-loop adapter can be linked to an end of a linear, double-stranded nucleic acid. For example, where stem-loop adapters are joined to the ends of a double-stranded target nucleic acid fragment, such that there are no terminal nucleotides (e.g., any gaps have been filled and ligated, using a polymerase and ligase, respectively), the resulting molecule lacks terminal nucleotides, instead bearing a single-stranded loop at each end.

The target nucleic fragment may be ligated to circularizable adapters. In this respect, fragments comprising the target sequence may be circularized by self-circularization of compatible structures on either side of the fragment (which may result from adapter ligation or as a result of restriction enzyme digestion of ligated adapters) or circularized by hybridization to a selector probe that is complementary to the ends of the desired fragment. Extension and a final step of ligation creates a covalently closed circular, optionally double-stranded, polynucleotide.

It is understood herein that the nucleic acid sample comprises at least one target nucleic acid fragment. Put differently, the nucleic acid sample thus may comprise 1, 2, 3, 4, 5, 6, 7, 8, 9, 10 or more target nucleic acid fragments, such as at least about 50, 100, 150, 200, 250, 300, 350, 400, 450, 500, 750, 1000 or more target nucleic acid fragments, wherein preferably each target nucleic acid fragment within the sample has a distinct sequence. The method of the invention may provide for a simultaneous enrichment of these target nucleic acid fragments from a nucleic acid sample. Therefore optionally, in step b) of the method of the invention, multiple sets of at least a first and second gRNA-CAS complexes are added for enrichment, isolation or sequencing of multiple target nucleic acid fragments from a nucleic acid sample. Preferably, these multiple sets of a first and second gRNA-CAS complexes may comprise the same CRISPR-nuclease, but may differ in their gRNA. For example, for each target nucleic acid fragment, two distinct gRNA molecules may be used, e.g. one gRNA is incorporated in the first gRNA-CAS complex another gRNA is incorporated in the second gRNA-CAS complex. For e.g. at least about 50, 100, 150, 200, 250, 300, 350, 400, 450, 500, 750, 1000 or more target nucleic acid fragments, preferably at least about 50, 100, 150, 200, 250, 300, 350, 400, 450, 500, 750, 1000 or more sets of gRNA molecules, preferably at least about 100, 200, 300, 400, 500, 600, 700, 800, 900, 1000, 1500, 2000 or more different gRNA molecules may be used in the method of the invention. As indicated herein, preferably, at least the sequences targeted by the sets of gRNA-CAS complexes shows sequence identity to the reference sequences used in step e) of the method of the invention, or a specific subset thereof.

Optionally, the method of the invention is multiplexed, i.e. applied simultaneously for multiple nucleic acid samples, such as for at least about 10, 20, 30, 40, 50, 60, 70, 80, 90, 100, 500, 1000 or more nucleic acid samples. The method may be performed in parallel for multiple samples, wherein “in parallel” is to be understood herein as substantially simultaneously but each sample being processed in a separate reaction tube or vessel. In addition or alternatively, one or more steps of the method of the invention may be performed on pooled samples. In order to trace back the enriched, isolated and/or sequenced fragment to the originating sample, the fragments may be tagged with an identifier prior to pooling the samples. Such identifier can be any detectable entity, such as, but not limited to, a radioactive or fluorescent label, but preferably is a particular nucleotide sequence or combination of nucleotide sequences, preferably of defined length. In addition or alternatively, the samples can be pooled using a clever pooling strategy, such as, but not limited to, a 2D and 3D pooling strategy, such that after pooling each sample is encompassed in at least two or three pools, respectively. A particular target fragment can be traced back to the originating sample by using the coordinates of the respective pools comprising the particular enriched, isolated and/or sequenced target fragment.

The nucleic acid sample of the method of the invention may be from any source, e.g. human, animal, plant, microorganism, and maybe of any kind, e.g. endogenous or exogenous to the cell, for example genomic DNA, chromosomal DNA, artificial chromosomes, plasmid DNA, or episomal DNA, cDNA, RNA, mitochondrial, or of an artificial library such as a BAC or YAC or the like. The DNA may be nuclear or organellar DNA. Preferably, the DNA is chromosomal DNA, preferably endogenous to the cell.

In step a) of the method of the invention, a sample is provided. The sample comprises a nucleic acid molecule comprising the sequence of interest. Optionally, the sample comprises multiple nucleic acid molecules comprising a sequence of interest. In addition or alternatively, the sample may comprise one or more further nucleic acid molecules not comprising the sequence of interest.

The total amount of nucleic acid molecules present in the provided sample is preferably more than about 1 pg, 10 pg, 100 pg, 1 ng, 10 ng, 100 ng, 1 μg, 10 μg or more than about 100 μg. Alternatively, the amount of nucleic acid molecules present in the provided sample is preferably less than about 1 pg, 10 pg, 100 pg, 1 ng, 10 ng, 100 ng, 1 μg, 10 μg or less than about 100 μg. The amount of nucleic acid molecules present in the provided sample is preferably about 1 pg-100 μg, about 10 pg-10 μg, about 100 pg-1 μg or about 1 ng-100 ng,

For each target nucleic acid fragment, the total amount present in the provided sample is preferably about 0.01-100 pg, or 0.1-100 pg or 10-50 pg, preferably about 0.01 pg, 0.05 pg, 0.1 pg, 1 pg, 5 pg, 10 pg, 15 pg, 20 pg, 25 pg, 30 pg, 40 pg, 50 μg, 60 pg, 70 pg, 80 pg, 90 pg or 100 pg. The total amount of each target nucleic acid fragment may be at most about 0.0001%, 0.001%, 0.01%, 0.1%, 1% or 10% of the total amount of the nucleic acid molecules in the sample.

The sample may comprise more than one target nucleic acid fragment. Preferably, the total amount of all target nucleic acid fragments present in the provided sample is about 0.01-100 ng, or 0.1-100 ng or 10-50 ng, preferably about 0.01 ng, 0.05 ng, 0.1 ng, 1 ng, 5 ng, 10 ng, 15 ng, 20 ng, 25 ng, 30 ng, 40 ng, 50 ng, 60 ng, 70 ng, 80 ng, 90 ng or 100 ng. The total amount of all target nucleic acid fragments may be at least 0.0001%, 0.001%, 0.01%, 0.1%, 1%, 10%, 20% or 30% of the total amount of the nucleic acid molecules in the sample and/or at most about 30%, 20%, 10%, 1%, 0.1%, 0.01%, 0.001% or 0.0001% of the total amount of the nucleic acid molecules in the sample. Preferably, the total amount of all target nucleic acid fragments is about 0.001%-30%, or 0.01%-20% or 0.1-10% of the total amount of the nucleic acid molecules in the sample.

Optionally, the method of the invention further comprises an amplification step. Preferably, such amplification step is after step e) of the method of the invention.

In a further aspect, the invention provides for a kit of parts for a method as defined herein above. Preferably, said kit comprises at least one of:
one or more vials comprising at least a first and second gRNA-CAS complex as defined herein;
one or more vials comprising at least a first and second gRNA for complexing with a CRISPR-CAS protein to form a gRNA-CAS complex, and a further vial comprising said CRISPR-CAS protein;
a further vial comprising one or more exonucleases for degrading a non-target nucleic acid; and
optionally a vial comprising one or more restriction enzymes for degrading non-target nucleic acid;
optionally one or more vials comprising reagents, such as sequencing adapter, for nanopore sequencing, preferably for nanopore selective sequencing.

Optionally, the kit further comprises one or more adapters as defined herein, either with the one or more vials indicated herein above or in separate vials. Preferably, the kit comprises at least 2, 4, 10, 20, or 50 vials comprising one or more gRNAs as defined herein. Preferably, the volume of any of the vials within the kit do not exceed 100 mL, 50 mL, 20 mL, 10 mL, 5 mL, 4 mL, 3 mL, 2 mL or 1 mL.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

Finally, the provided is for the use of at least a first and second gRNA-CAS complex or a kit or parts as defined herein for enrichment of at least one target nucleic acid fragment from a nucleic acid sample. More in particular, provided is for the use of at least a first and second gRNA-CAS complex for protecting a target nucleic acid fragment against exonuclease degradation.

### EXAMPLES

### Example

### Material, Methods and Results

In order to investigate the method on crop DNA, sgRNAs were designed to target 814 loci in Melon Vedrantais genomic DNA, each of these targets having a length of 5.1 to 6.9 kbp. For each target, a couple of at least two sgRNAs were designed to target both the up- and downstream regions of approximately 100 to 1000 bp flanking each target, wherein each sgRNA comprises a 20 nts-long guide sequence which is unique within the genome.

S. pyrogenes Cas9 nuclease (6.5 pmol; New England Biolabs Inc.) was complexed with the sgRNA (6.5 pmol) in a volume of 10 μl of 1×Cas9 Nuclease reaction buffer (New England Biolabs Inc.) and pre-incubated for 10 minutes at 25° C. Subsequently 1 μg of DNA was added to a total volume of 30 μl. The mixture was incubated for 1 hour at 37° C. Unprotected fragments were removed through incubation with Exonuclease V. For this the following components were added to 15 μl of the Cas9 reaction, 0.5 μl 10×NEB 3.1 buffer, 2.0 μl 10 mM ATP (New England Biolabs), 1.5 μl 10 U/μl ExoV exonuclease (New England Biolabs) and 1 μl nuclease-free water. The resulting 20 μl reaction mixture was incubated at 37° C. for 60 minutes. The proteins were inactivated through incubation for 30 minutes at 70° C.

The above mentioned reaction, i.e. Cas9-cleavage and protection of targets and digestion of unprotected non-targets by Exonuclease, was performed 3× per sample using 1 μg of DNA for each sample.

After the Exonuclease V digestion, all reactions for the same sample were pooled resulting in a total of 120 μl reaction mixture. To digest the exonuclease by hydrolyzing peptide bonds, 6 μl 20 mg/ml Proteinase K (Roche) was added to the 120 μl total reaction mixture and incubated for 15 minutes at room temperature. Inactivation of the remaining Proteinase K was performed at 75° C. for 20 minutes. The reactions were purified using the Ampure PB bead solution (Pacific Biosciences) with a ratio of 0.5× beads to sample. After binding to a magnet, the beads were washed twice with 70% ethanol. Beads were dried for 1 minute and the bound DNA was eluted in 24 μl nuclease-free water. The eluted DNA was analyzed using the FEMTO Pulse (Advanced Analytical).

Eluted DNA was used for barcoded sequencing library preparation using barcoding kits EPX-NBD1014 and EXP-NBD114 in combination with library preparation kit SQK-LSK109 (Oxford Nanopore Technologies) for sequencing using the Oxford Nanopore MinION system. In parallel, a sequencing library from Vedrantais genomic DNA (WGS samples) was also prepared using sequencing library preparation kit SQK-LSK109 (Oxford Nanopore Technologies). Library preparation and sequencing was performed according manufacturers specifications. In brief, barcoding adapters and subsequently sequencing adapters were ligated to the fragment-enriched samples after end-polishing and A-addition. For the WGS samples, sequencing adapters were ligated directly after end-polishing and A-addition.

Nanopore selective sequencing was performed using the Read Until as also described in Payne et al. 2020 (Nanopore adaptive sequencing for mixed samples, whole exome capture and targeted panels, Feb. 3, 2020; DOI: 10.1101/2020.02.03.926956), at standard settings wherein the sequencer was programmed to sequence the 814 target loci targeted by the sgRNA as indicated herein above, both for the targeted enriched samples by Cas9-cleavage and protection of targets and digestion of unprotected non-targets by Exonuclease (“TarSeq samples”), as well as for the whole genome samples (“WGS samples”). Standard settings were applied.

Obtained sequence reads were quality filtered using manufacturers setting and passed reads were mapped against the whole genome reference sequence of melon Vedrantais. For mapping the reads, minimap2.11-r797 was used with standard settings. From the mapped reads, only those that had a single mapping position were used for further analysis.

### CONCLUSION

The target enrichment using CRISPR-system nuclease complex for selecting fragments by protecting against exonuclease digestion highly boosts subsequent enrichment by nanopore selective sequencing, resulting in an over 50 times enrichment of targeted regions of interest.

TABLE 1
Nanopore sequencing of whole genomes (WGS) or an enriched library prepared using
CRISPR-Cas9 selection of target sequences in combination with exonuclease degradation
of non-target sequences (TarSeq) by standard nanopore sequencing (Standard run)
or by nanopore selective sequencing (Read Until run). Average depth on target,
Average depth whole genome (WG, average nucleotide depth outside the target regions)
and fold enrichment target over WG (average fold increase of nucleotide depth on
target) indicated herein are average values per target per sample.

| | WGS | WGS | TarSeq | TarSeq |
|---|---|---|---|---|
| | (Standard run) | (Read Until run) | (Standard run) | (Read Until run) |
| Average depth on target | 31.83 | 37.60 | 8.1 | 20.7 |
| Average depth WG | 29.47 | 4.60 | 0.71 | 0.34 |
| Enrichment | 1.08x | 8.17x | 11.41x | 60.17x |

1. A method for sequencing of a target nucleic acid fragment from a sample comprising a nucleic acid molecule, wherein the target nucleic acid fragment comprises a sequence of interest, and wherein the method comprises the steps of:
a) providing the sample comprising the nucleic acid molecule, wherein the nucleic acid molecule comprises the sequence of interest;
b) cleaving the nucleic acid molecule with at least a first and a second gRNA-CAS complex, thereby generating the target nucleic acid fragment comprising the sequence of interest that is protected against exonuclease cleavage, and at least one non-target nucleic acid fragment;
c) contacting the cleaved nucleic acid molecules obtained in step b) with an exonuclease and allowing the exonuclease to digest the at least one non-target nucleic acid fragment;
d) optionally purifying the target nucleic acid fragment comprising the sequence of interest from the digest obtained in step c); and
e) sequencing the (non-digested and optionally purified) target nucleic acid fragment by nanopore selective sequencing.

2. The method according to claim 1, wherein the method does not comprise a further step of protecting the target nucleic acid fragment, or the ends of the target nucleic acid fragment, prior to exonuclease digestion in step c).

3. The method according to claim 1, wherein at least one of
i) step b) is performed by incubating the first and second gRNA-CAS complex and the nucleic acid molecule together for about 1 min to about 18 hours, at about 10-90° C.; and
ii) step c) is performed by incubating the cleaved nucleic acid molecule with the exonuclease for about 1 minute to about 12 hours, at about 10-90° C.

4. The method according to claim 1, wherein at least one of the first and second gRNA-CAS complex comprises a Cas9 protein.

5. The method according to claim 1, wherein the at least one of the first and second gRNA-CAS complex comprises a sgRNA.

6. The method according to claim 1, wherein at least one of the first and second gRNA-CAS complex comprises a crRNA and a tracrRNA as separate molecules.

7. The method according to claim 1, wherein at least one of the first and second gRNA-CAS complex is capable of inducing a DSB.

8. The method according to claim 1, wherein both the first and the second gRNA-CAS complex are capable of inducing a DSB.

9. The method according to claim 1, wherein in step b) at least one of the first and second gRNA-CAS complex nicks one strand of the nucleic acid molecule, and wherein the nucleic acid molecule is contacted with at least a third gRNA-CAS complex that nicks the complement strand at substantially the complementary position of the position nicked by said first or second gRNA-CAS complex.

10. The method according to claim 1, wherein the method comprises a step of adapter ligation prior to sequencing step e.

11. The method according to claim 10, wherein the adapters are sequence adapters.

12. The method according to claim 1, wherein the method is performed in parallel for multiple nucleic acid samples.

13. The method according to claim 1, wherein the nucleic acid molecule is genomic DNA.

14. The method according to claim 1, wherein the nucleic acid molecule is a nucleic acid molecule obtainable from a plant, animal, human or microorganism.

15. A kit of parts for enrichment of a target nucleic acid fragment from a nucleic acid molecule comprising:
at least a first and second gRNA-CAS complex as defined in claim 1;
an exonuclease; and
reagents for nanopore sequencing.

16. (canceled)

17. The method of claim 3, wherein step b) is performed for about 60 minutes.

18. The method of claim 3, wherein step b) and/or step c) is performed at about 37° C.

19. The method of claim 3, wherein step c) is performed for about 30 minutes.

20. The method of claim 10, wherein the step of adapter ligation is following contacting step c) or optionally purifying step d).
